(19) United States
(12) Patent Application Publication
(10) Pub. No.: US 2025/0177077 A1
(43) Pub. Date: Jun. 5, 2025

(54) Packaging for Terminal Sterilization

(71) Applicant: Becton, Dickinson and Company, Franklin Lakes, NJ (US)

(72) Inventors: Sameer Upadhyaya, Allendale, NJ (US); Vikas Patel, Sandy, UT (US); Ryan Smart, Wauconda, IL (US); Hariharan Iyer, Thane (IN); Zachary Komro, Wheeling, IL (US); Supradeep Vankayala, Bangalore (IN); Amit Limaye, Wayne, NJ (US)

(21) Appl. No.: 18/842,223

(22) PCT Filed: Mar. 2, 2023
(86) PCT No.: PCT/US23/14351

§ 371 Date: Aug. 28, 2024

Related U.S. Application Data
(60) Provisional application No. 63/315,786, filed on Mar. 2, 2022.

(51) Int. Cl. A61B 50/33; A61B 50/30

(52) U.S. Cl. CPC A61B 50/33; A61B 2050/3008; A61B 2050/3015; A61B 2050/314

(57) ABSTRACT

Provided herein is a terminally-sterilizable package for a medical device having a first flexible web having a proximal end, a distal end, and at least one side therebetween defining a first perimeter, and a second flexible web having a proximal end, a distal end, and at least one side therebetween defining a second perimeter at least partially overlapping with the first perimeter at one or more sealing regions, the second flexible web removably attached to the first flexible web, the first flexible web and the second flexible web defining a compartment configured to hold the medical device, wherein the one or more sealing regions define a tortuous path fluidly connecting the compartment with an exterior of the package.

### CROSS-REFERENCE TO RELATED APPLICATION

The present application claims priority to U.S. Provisional Patent Application No. 63/315,786, entitled “Packaging for Terminal Sterilization” filed Mar. 2, 2022, the entire disclosure of which is hereby incorporated by reference in its' entirety.

### BACKGROUND OF THE INVENTION

### Field of the Invention

The present invention relates generally to packaging for medical devices, and, in particular embodiments or aspects, to terminally-sterilizable packaging.

### Description of Related Art

Medical devices, and packaging for the same, require sterilization. An approach has been to terminally sterilize the device and packaging once the device has been packaged. However, there are shortcomings to traditional materials used for the packaging.

Specifically, most plastic films, which are transparent or substantially transparent so to allow for visualization of the medical device within the packaging, are not gas permeable, thus preventing use of gaseous sterilization methods, such as ethylene oxide (EtO), hydrogen peroxide, ozone, nitrous oxide, and steam. To allow for gaseous sterilization methods, gas-permeable materials, such as paper-like materials, or TYVEK, have been used. However, such materials are typically not transparent, and do not allow visualization of the device inside the packaging. To address this shortcoming, packaging formed of more than one material has been used, but such packaging is typically more expensive and complicated to produce. Thus, there remains a need in the art for a terminally-sterilizable packaging for medical device.

### SUMMARY OF THE INVENTION

Provided herein is a terminally-sterilizable package for a medical device, including a first flexible web having a proximal end, a distal end, and at least one side therebetween defining a first perimeter; and a second flexible web having a proximal end, a distal end, and at least one side therebetween defining a second perimeter at least partially overlapping with the first perimeter at one or more sealing regions, the second flexible web removably attached to the first flexible web, the first flexible web and the second flexible web defining a compartment configured to hold the medical device, wherein the one or more sealing regions define a tortuous path fluidly connecting the compartment with an exterior of the package.

In accordance with an embodiment of the present invention, the one or more sealing regions are arranged between the first flexible web and the second flexible web at the proximal ends thereof.

In accordance with an embodiment of the present invention, the one or more sealing regions are arranged between the first flexible web and the second flexible web at the distal ends thereof.

In accordance with an embodiment of the present invention, the one or more sealing regions are arranged between the first flexible web and the second flexible web at the one or more sides thereof.

In accordance with an embodiment of the present invention, the one or more sealing regions are arranged at more than one location of overlap between the first perimeter and the second perimeter.

In accordance with an embodiment of the present invention, the first flexible web and the second flexible web are formed of the same material.

In accordance with an embodiment of the present invention, the material includes one or more flexible thermoformable plastics.

In accordance with an embodiment of the present invention, the flexible thermoformable plastics includes one or more of nylon-based films, polyethylene, and/or ethyl vinyl acetate.

In accordance with an embodiment of the present invention, the tortuous path includes one or more curves.

In accordance with an embodiment of the present invention, the tortuous path includes one or more right angles.

In accordance with an embodiment of the present invention, the tortuous path includes a honeycomb arrangement.

In accordance with an embodiment of the present invention, the tortuous path fluidly connects the compartment with the exterior of the package at a plurality of locations along the first perimeter and the second perimeter.

In accordance with an embodiment of the present invention, the tortuous path is arranged in substantially all of the overlap between the first perimeter and the second perimeter.

In accordance with an embodiment of the present invention, the first flexible web and the second flexible web includes one or more overlapping regions extending radially inwards into the compartment.

In accordance with an embodiment of the present invention, the tortuous path is arranged at least in the one or more inwardly-extending regions.

Also provided herein is a terminally-sterilizable package for a medical device, including a tray having a closed distal end; an open proximal end; a sidewall arranged between the closed distal end and the open proximal end, the closed distal end and sidewall defining a well configured to receive the medical device therein; and a flange arranged about a perimeter of the open proximal end of the tray; and a flexible web arranged at the open proximal end of the tray and at least partially overlapping the flange at one or more sealing regions, wherein the one or more sealing regions define a tortuous path fluidly connecting the well with an exterior of the package.

In accordance with an embodiment of the present invention, the one or more sealing regions are arranged at more than one location of overlap between the flange and the flexible web.

In accordance with an embodiment of the present invention, the tray and/or the flexible web includes one or more flexible thermoformable plastics.

In accordance with an embodiment of the present invention, the flexible thermoformable plastics includes one or more of nylon-based films, polyethylene, and/or ethyl vinyl acetate.

In accordance with an embodiment of the present invention, the tortuous path includes one or more curves.

In accordance with an embodiment of the present invention, the tortuous path includes one or more right angles.

In accordance with an embodiment of the present invention, the tortuous path includes a honeycomb arrangement.

In accordance with an embodiment of the present invention, the tortuous path fluidly connects the well with the exterior of the package at a plurality of locations along the perimeter of the well.

In accordance with an embodiment of the present invention, the tortuous path is arranged in substantially all of the overlap between the first perimeter and the second perimeter.

In accordance with an embodiment of the present invention, the flange and the flexible web include one or more overlapping regions extending radially inwards into the open proximal end of the tray.

In accordance with an embodiment of the present invention, the tortuous path is arranged at least in the one or more inwardly-extending regions.

Also provided herein is a terminally-sterilizable package for a medical device, including a tray having a closed distal end; an open proximal end; a sidewall arranged between the closed distal end and the open proximal end, the closed distal end and sidewall defining a well configured to receive the medical device therein; and a flange, connected to the sidewall and arranged about a perimeter of the open proximal end of the tray, the sidewall and the flange comprising one or more cutouts; a flexible web arranged at the open proximal end of the tray and at least partially overlapping the flange; and one or more plugs arranged in the one or more cutouts in the sidewall and the flange, wherein the one or more plugs define a tortuous path fluidly connecting the well with an exterior of the package.

In accordance with an embodiment of the present invention, the cutout and the plug have a substantially circular cross-sectional shape.

In accordance with an embodiment of the present invention, the cutout and the plug have a substantially quadrilateral cross-sectional shape.

In accordance with an embodiment of the present invention, the cutout and the plug have a substantially rectangular cross-sectional shape.

In accordance with an embodiment of the present invention, the cutout and the plug have a substantially trapezoidal cross-sectional shape.

In accordance with an embodiment of the present invention, the tray and/or the flexible web include one or more flexible thermoformable plastics.

In accordance with an embodiment of the present invention, the flexible thermoformable plastics include one or more of nylon-based films, polyethylene, and/or ethyl vinyl acetate.

In accordance with an embodiment of the present invention, the flexible web overlaps the flange at one or more sealing regions, and wherein the one or more sealing regions define a tortuous path fluidly connecting the well with an exterior of the package.

In accordance with an embodiment of the present invention, the tortuous path includes one or more curves.

In accordance with an embodiment of the present invention, the tortuous path includes one or more right angles.

In accordance with an embodiment of the present invention, the tortuous path includes a honeycomb arrangement.

Also provided herein is a terminally-sterilizable package for a medical device, including a first flexible web having a proximal end, a distal end, and at least one side therebetween defining a first perimeter; and a second flexible web having a proximal end, a distal end, and at least one side therebetween defining a second perimeter at least partially overlapping with the first perimeter at one or more sealing regions, the second flexible web removably attached to the first flexible web, the first flexible web and the second flexible web defining a compartment configured to hold the medical device, wherein one or both of the first flexible web and the second flexible web comprises one or more filters configured to allow ingress of a sterilizing gas into the compartment.

In accordance with an embodiment of the present invention, the first flexible web and the second flexible web are formed of the same material, and the package is configured to be recycled in single-stream recycling.

In accordance with an embodiment of the present invention, the material includes polypropylene or polyethylene.

In accordance with an embodiment of the present invention, the material includes low-density polyethylene or linear low-density polyethylene.

In accordance with an embodiment of the present invention, the one or more filters are is formed of the same material as the first flexible web and the second flexible web.

In accordance with an embodiment of the present invention, the package is configured to be sterilized by a sterilizing gas and/or irradiation.

Also provided herein is a terminally-sterilizable package for a medical device, including a flexible web having a proximal end, a distal end, and at least one side therebetween defining a first perimeter and a compartment configured to hold the medical device, at least one of the ends and/or sides defining a seam, wherein the seam defines a tortuous path fluidly connecting the compartment with an exterior of the package.

In accordance with an embodiment of the present invention, the flexible web includes polypropylene or polyethylene, and wherein the package is configured to be recycled in single-stream recycling.

In accordance with an embodiment of the present invention, the flexible web includes low-density polyethylene or linear low-density polyethylene.

In accordance with an embodiment of the present invention, the tortuous path includes one or more curves.

In accordance with an embodiment of the present invention, the tortuous path includes one or more right angles.

In accordance with an embodiment of the present invention, the tortuous path includes a honeycomb arrangement.

In accordance with an embodiment of the present invention, the tortuous path fluidly connects the compartment with the exterior of the package at a plurality of locations along the seam.

In accordance with an embodiment of the present invention, the package is configured to be sterilized by a sterilizing gas and/or irradiation.

### BRIEF DESCRIPTION OF THE DRAWINGS

FIG. 1 is a schematic view of a terminally-sterilizable package for a medical device according to non-limiting embodiments or aspects;

FIG. 2 is a schematic view of a terminally-sterilizable package for a medical device according to non-limiting embodiments or aspects;

FIG. 3 is a schematic view of a terminally-sterilizable package for a medical device according to non-limiting embodiments or aspects;

FIG. 4 is a schematic view of a terminally-sterilizable package for a medical device according to non-limiting embodiments or aspects;

FIGS. 5A-5M are schematic views of a terminally-sterilizable package for a medical device according to non-limiting embodiments or aspects;

FIG. 6 is a schematic view of a terminally-sterilizable package for a medical device according to non-limiting embodiments or aspects;

FIGS. 7A-7C are schematic views of a terminally-sterilizable package for a medical device according to non-limiting embodiments or aspects;

FIG. 8 is a schematic view of a terminally-sterilizable package for a medical device according to non-limiting embodiments or aspects; and

FIG. 9 is a graph showing diffusion distance over time for particles having varying diameters.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

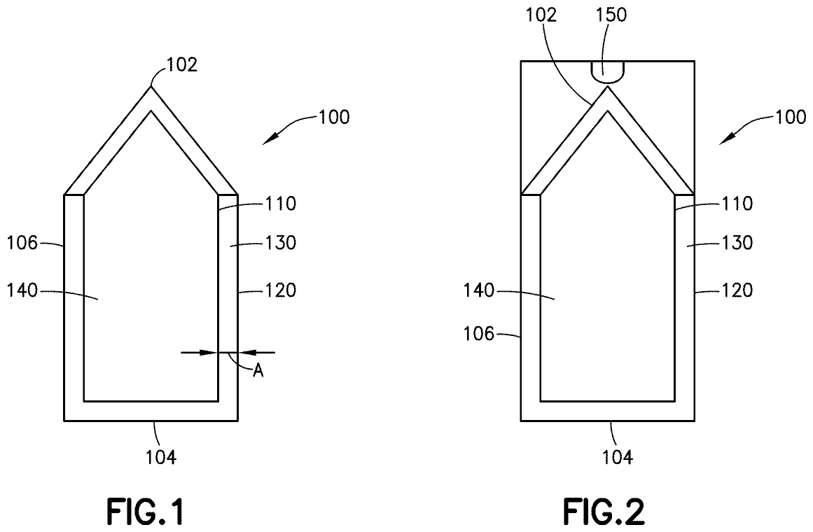

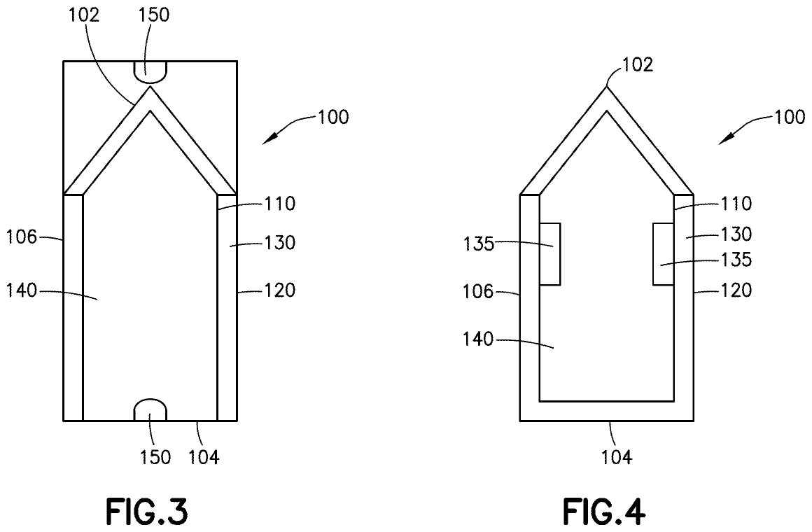

Referring to FIGS. 1-4, a terminally-sterilizable package 100 for a medical device according to non-limiting embodiments or aspects has a proximal end 102, a distal end 104, and one or more sides 106. As used herein, the term “terminally-sterilizable” refers to a means of eliminating or killing microorganisms present on a surface of the packaged medical device and/or the packaging in which the device is contained, after the device has been sealed within the packaging, in order to achieve asepsis or a sterile microbial environment. Terminal sterilization can be achieved by applying heat, chemicals, irradiation/radiation, high pressure, filtration, or combinations thereof. Chemical sterilization includes sterilization with gases such as ethylene oxide (EtO), hydrogen peroxide gas, ozone, nitrous oxide, and/or steam, liquids such as chlorine bleach, iodine, glutaraldehyde and formaldehyde, ortho-phthaladehyde (OPA), hydrogen peroxide, peracetic acid, sodium hydroxide, silver, and cobalt. Radiation sterilization involves the use of radiation such as electron beams (E-beam), x-rays, gamma rays, or subatomic particles. In non-limiting embodiments or aspects, the terminal sterilization is a gaseous sterilization, including EtO, hydrogen peroxide gas, ozone, nitrous oxide, and/or steam. The terminally-sterilizable package 100 can be, for example, a blister package. In non-limiting embodiments or aspects, terminally-sterilizable package 100 includes a first flexible web 110 and, optionally, a second flexible web 120, defining a compartment 140 configured to receive the medical device. Each of the webs has a proximal end and a distal end, and at least one side therebetween defining respective perimeters. First flexible web 110 and second flexible web 120 overlap at least partially at one or more regions along their respective perimeters. In non-limiting embodiments, a width of the region of overlap (denote A in FIG. 1) is between about ⅜ of an inch and about 1 inch, optionally between about ⅜ of an inch and about ¾ of an inch, all values and subranges therebetween inclusive. Within these one or more regions of overlap are one or more sealing regions 130. In non-limiting embodiments, sealing regions 130 may be about 2-7, about 3-6, and/or about 4-5 mm wide, all values and subranges therebetween inclusive. In non-limiting embodiments, a total length of a perimeter of the sealing regions 130 may be at least 300 mm, optionally at least 350 mm, optionally at least 374 mm, all values and subranges therebetween inclusive. In non-limiting embodiments or aspects, one or both of the first flexible web 110 and the second flexible web 120 include an inwardly-protruding section 135, which extends into compartment 140 from the respective perimeters thereof. In non-limiting embodiments or aspects, the one or more sealing regions 130 are arranged to at least partially include such inwardly-protruding sections 135. In non-limiting embodiments or aspects, a width of the one or more inwardly-protruding sections 135 is about ¾ of an inch. In non-limiting embodiments or aspects, one or both of the first flexible web 110 and second flexible web 120 can include one or more thumb notches 150 to allow a user to separate the webs to access the compartment and the medical device held therein.

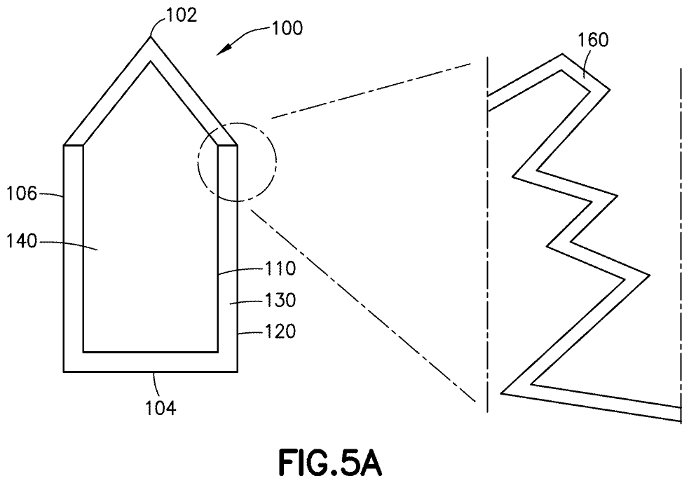

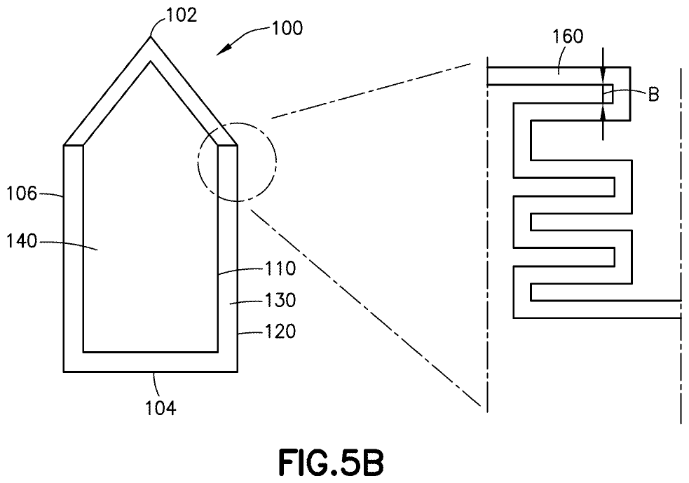

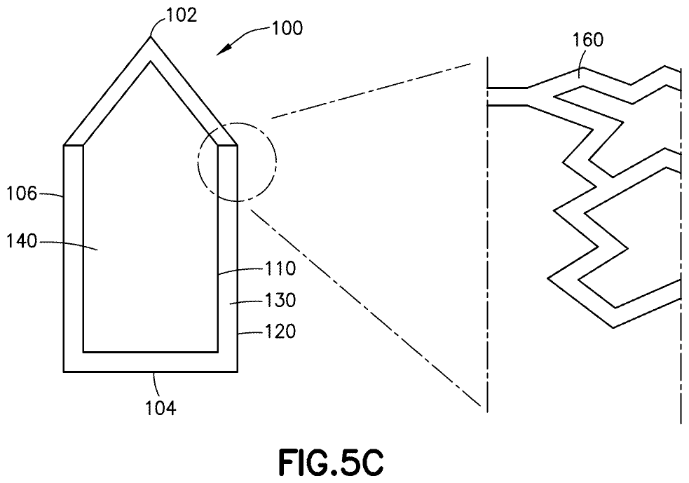

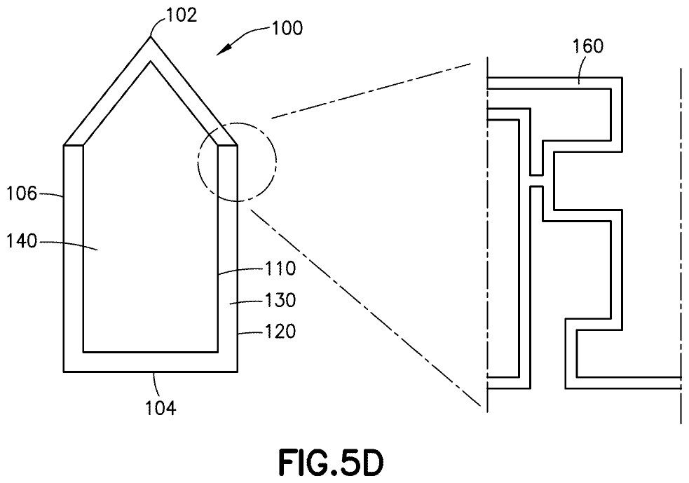

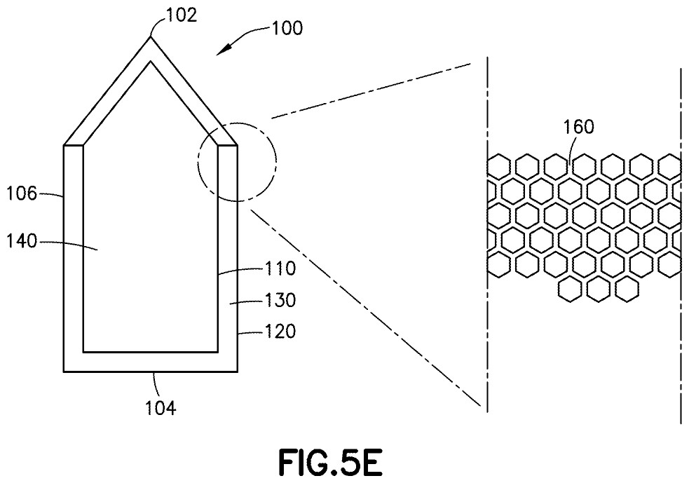

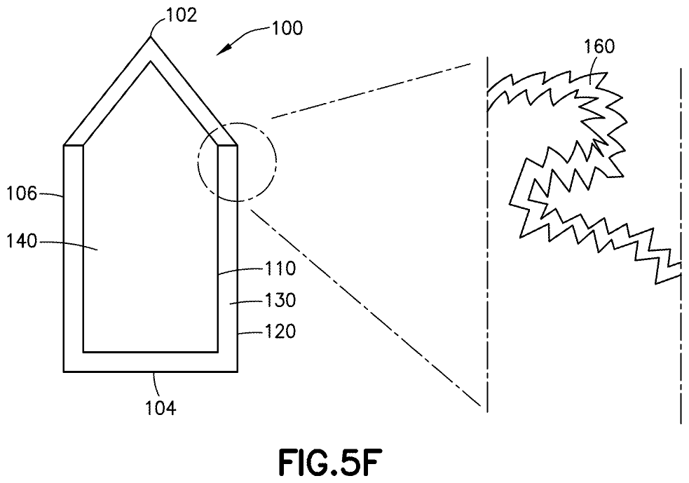

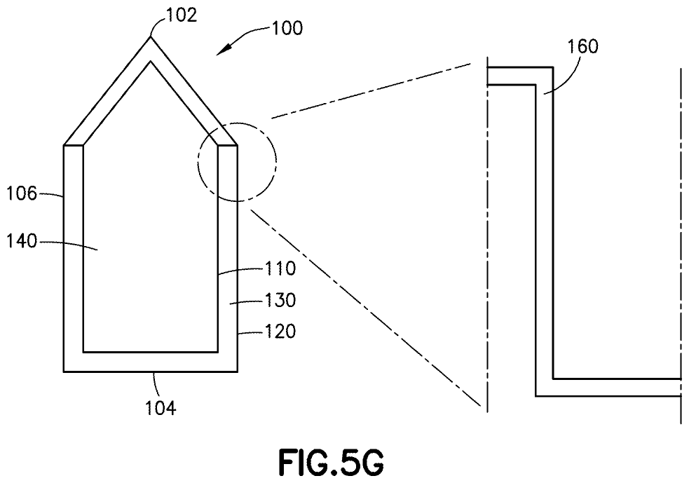

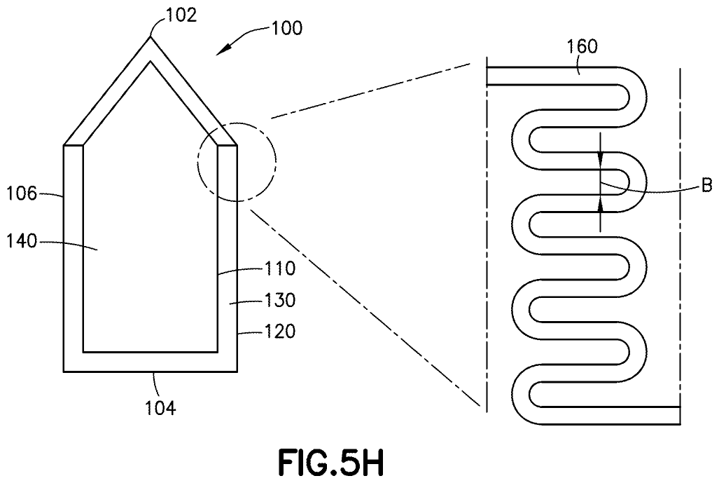

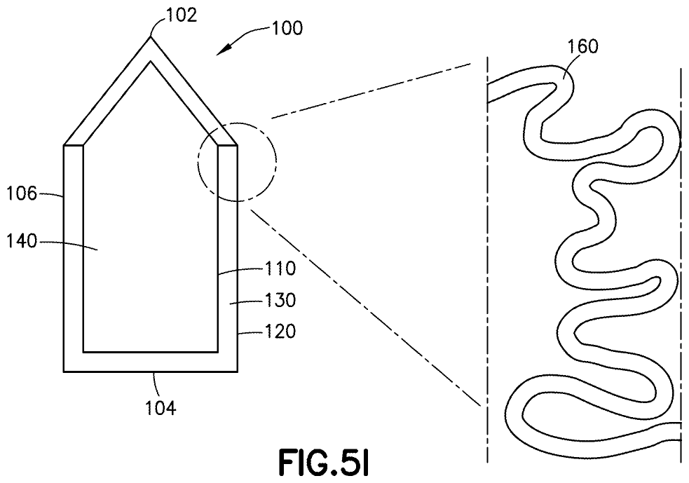

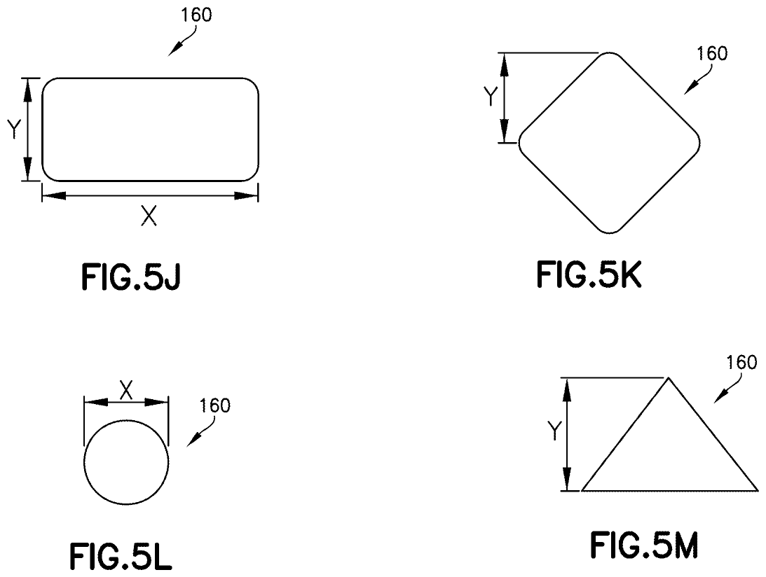

In non-limiting embodiments or aspects, these one or more sealing regions 130 define one or more tortuous paths 160 (for example, those shown in FIGS. 5A-5I) that fluidly connect the compartment 140 and the exterior of the package 100. The one or more sealing regions 130, including the one or more tortuous paths 160, can be arranged at one or more locations about the perimeter of the package, including the proximal end 102, distal end 104, and/or side 106. This fluid connectivity allows sterilization gases, such as those described herein, to reach the medical device, while also prohibiting ingress of microorganisms.

First and second flexible webs 110, 120 can be formed of one or more layers of any material known to those of skill in the art to be useful for packaging. In non-limiting embodiments or aspects, the material is substantially or completely transparent. In non-limiting embodiments or aspects, the material is a flexible thermoformable plastic film. In non-limiting embodiments or aspects, the film is multi-layered. In non-limiting embodiments or aspects, the primary component of the film is a layer of approximately 15-30% Nylon, while the remaining layers can comprise substances including, but not limited to, polyethylene. In non-limiting embodiments or aspects, the material is ethylene vinyl acetate (EVA), optionally up to about 30% EVA. In non-limiting embodiments or aspects, first and/or second flexible webs 110, 120 are formed of a radiation-stable material. In non-limiting embodiments or aspects, first and/or second flexible webs 110, 120 are formed of polyethylene, low-density polyethylene, linear low-density polyethylene, polypropylene, and/or stabilized polypropylene. In non-limiting embodiments or aspects, the first flexible web 110 and the second flexible web 120 are formed of the same material or class of materials, and no sealant or adhesive is required or is included. In such embodiments, a sheet of the material may be fused onto itself to create a seam, for example by heat sealing, laser ablation, or other like techniques known to those of skill in the art. These seams, like sealing regions 130 described above, may define one or more tortuous paths (for example, those shown in FIGS. 5A-5I) that fluidly connect the compartment and the exterior of the package 100. Use of a single material (or single class of materials) for first and/or second flexible webs 110, 120, allows for package 100 to be easily recyclable in single-stream recycling.

With reference to FIGS. 5A-5M, shown are non-limiting embodiments or aspects of tortuous paths 160 that fluidly connect the compartment 140 of the package 100 and the exterior of the package, to allow ingress of sterilizing gases. The one or more tortuous paths 160 can be of any shape and/or geometry, have any diameter, and can include one or more curves, right angles, acute angles, and/or oblique angles. In non-limiting embodiments, one or more tortious paths 160 have a circular, triangular, and/or quadrilateral geometry, for example as shown in FIGS. 5J-5M. In non-limiting embodiments, the quadrilateral geometry may be a square or rectangular shape. In non-limiting embodiments, tortious paths 160 are arranged about a perimeter of package 100. In non-limiting embodiments, one or more tortious paths 160 have a diameter of no greater than 1 mm, optionally no greater than 250 μm, optionally no greater than 3.0 μm, all values and subranges therebetween inclusive. In non-limiting embodiments, for example as shown in FIGS. 5J-5M, tortious path 140 may have a rectangular (FIG. 5J), square (FIG. 5K), circular (FIG. 5L), and/or triangular (FIG. 5M) cross-sectional geometry. As noted, tortious path 140 may have any suitable diameter; however, in non-limiting embodiments, tortious path 140 has a width (x) of about 1 mm and a height (y) of about 0.5 mm, a height (y) of about 1 mm, and/or a diameter (x) of about 0.26 mm.

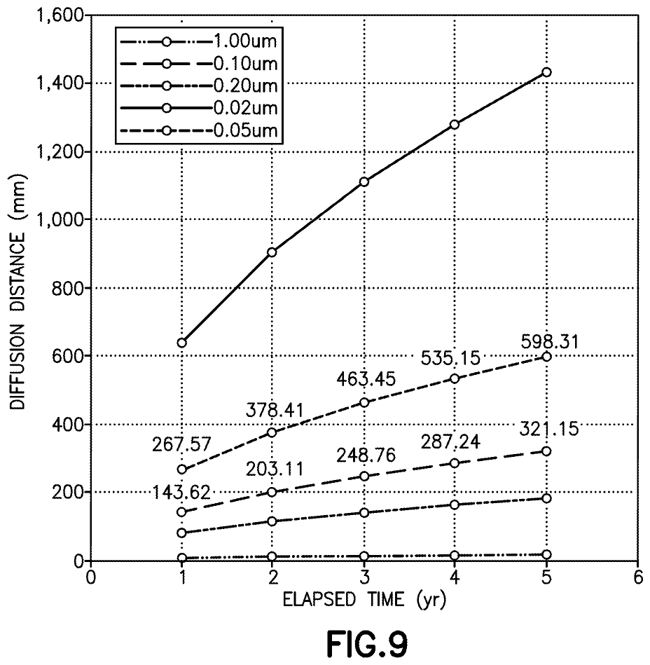

In non-limiting embodiments or aspects, a distance between curves and/or angles (e.g., B in FIGS. 5B and 5H) can be in the range of 1-5 mm, optionally about 4.5 mm, all values and subranges therebetween inclusive. In non-limiting embodiments, one or more tortious paths includes at least one 90° angle, optionally a curved 90° angle. The one or more tortuous paths 160 can be of any length, can intersect with one another any number of times, and/or can open into the compartment 140 and/or the exterior of package 100 at one or more locations. In non-limiting embodiments or aspects, one or more tortuous paths 160 can be configured as a honeycomb. In non-limiting embodiments or aspects, one or more tortuous paths 160 can include one or more sawtooth regions, for example as shown in FIG. 5F. Those of skill will appreciate that one or more tortuous paths 160 can include any number and any combination of any arrangement/configuration of paths. Without wishing to be bound by the theory, the size of the microorganism to be prevented from ingressing may correspond to a length of the one or more tortious paths 160, where a microorganism having a smaller diameter may require a longer tortious path. See, for example, Tsuda et al., Particle Transport and Deposition: Basic Physics of Particle Kinetics. Compr. Physiol. 2013, 3 (4): 1437-1471; Louten, Virus Structure and Classification. Ess. Hum. Virol. 2016, 19-29; and Pui et al., Experimental Study of Particle Deposition in Bends of Circular Cross Section. Aero. Sci. & Tech. 1987, 3001-315; and Zhang et al., Experimental Measurement and Numerical Study of Particle Deposition in Highly Idealized Mouth-Throat Models. Aero. Sci. & Tech. 2006, 5:361-372, the contents of which are incorporated herein by reference in their entirety. FIG. 9, shows a graph detailing diffusion distance of particles of various diameters over a number of years, which supports the length of tortious paths described above. One of skill may select an appropriate length of tortious path, knowing the particle diameter and diffusivity coefficient, based on the following equation:

$${L}_{D} \approx \sqrt{D \cdot t}$$

where D=a diffusivity coefficient (in cm²/s) and t=time (in sec).

In non-limiting embodiments, one or more tortious paths 160 may have a length of at least 10 mm, at least 20 mm, at least 100 mm, at least 200 mm, at least 300 mm, at least 400 mm, at least 500 mm, at least 600 mm, at least 700 mm, at least 1 cm, at least 5 cm, at least 10 cm, at least 30 cm, all values and subranges therebetween inclusive. In non-limiting embodiments, the one or more tortious paths 160 have a length of at least 20.5 mm, at least 321 mm, at least 600 mm, at least 10 cm, and/or at least 30 cm. In non-limiting embodiments, a tortious path length of at least 20.5 mm prevents bacteria ingress, a tortious path length of at least 321 mm prevents ingress of larger viruses (e.g., greater than 0.05 μm in diameter), and a tortious path of at least 600 mm prevents ingress of viruses having a diameter of 0.05 μm or less. In addition, a longer tortious path may increase the length of time that microorganisms are prevented from ingressing into compartment 140.

As would be appreciated by one of skill in the art, the varying characteristics of the tortious paths 160 described above may require differing terminal sterilization lengths, to ensure that the sterilizing medium may ingress to the compartment 140.

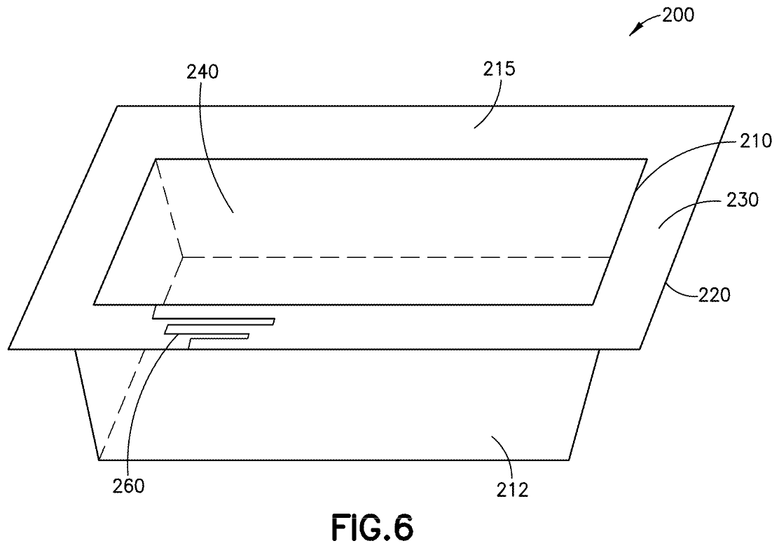

Turning to FIG. 6, a terminally-sterilizable package 200 for a medical device according to non-limiting embodiments or aspects has a first web 210 in the form of a tray, optionally a tray formed of a rigid material, having a closed distal end, an open proximal end, and a sidewall 212 arranged therebetween defining a well 240 configured to receive the medical device. First web 210 can also include a flange 215 arranged about the perimeter of the open proximal end thereof. Second flexible web 220 can be arranged at the open proximal end of first flexible web 210, at least partially overlapping flange 215 to create one or more sealing regions 230. In non-limiting embodiments or aspects, one or both of the flange 215 and the second flexible web 220 include an inwardly-protruding section (not shown), which extends into open proximal end of first web 210. In non-limiting embodiments or aspects, the one or more sealing regions 230 are arranged to at least partially include such inwardly-protruding sections. As described above, these one or more sealing regions 230 define one or more tortuous paths 260 that fluidicly connect the well and the exterior of the package. One or more tortious paths 260 may have any characteristic of tortious paths 160 described above. This fluid connectivity allows sterilization gases, such as those described herein, to reach the medical device, while also prohibiting ingress of microorganisms. As described above, and shown in non-limiting embodiments or aspects in FIGS. 5A-5I, one or more tortuous paths 260 can assume any useful configuration. The one or more tortuous paths 260 can be of any shape, and can include one or more curves, right angles, acute angles, and/or oblique angles. The one or more tortuous paths 260 can be of any length, can intersect with one another any number of times, and/or can open into the well 240 and/or the exterior of package 200 at one or more locations. In non-limiting embodiments or aspects, one or more tortuous paths 260 can be configured as a honeycomb. In non-limiting embodiments or aspects, one or more tortuous paths 260 can include one or more sawtooth regions, for example as shown in FIG. 5F. Those of skill will appreciate that one or more tortuous paths 260 can include any number and any combination of any arrangement/configuration of paths.

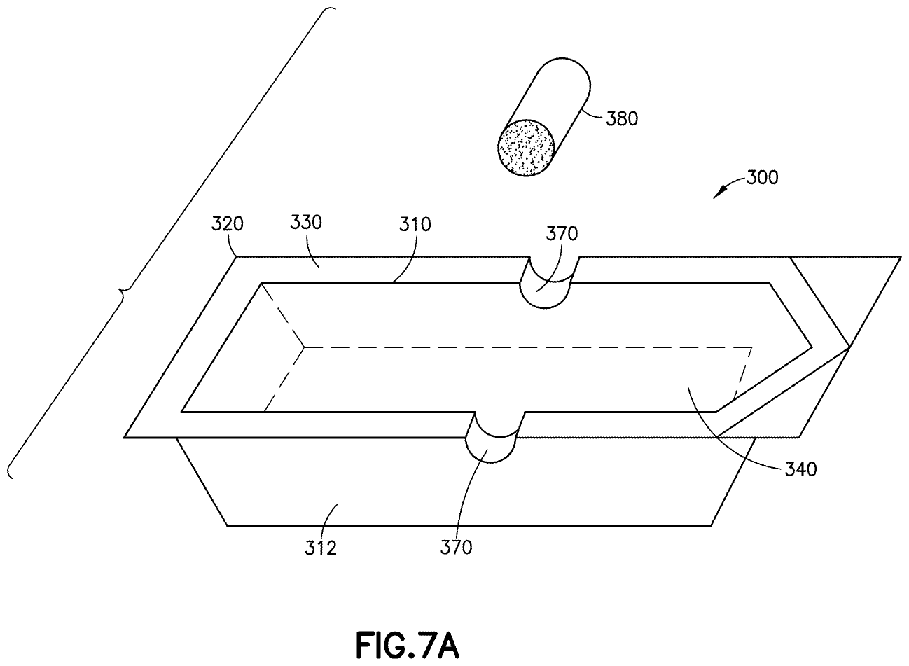

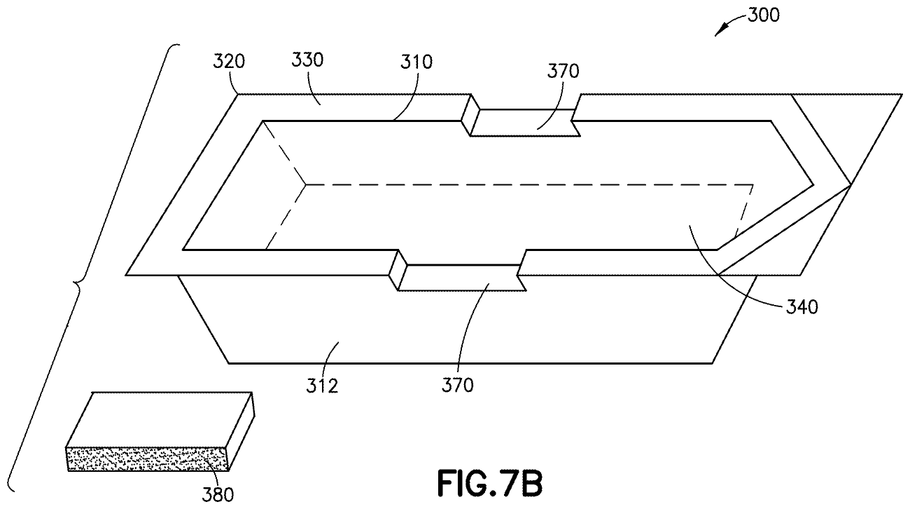

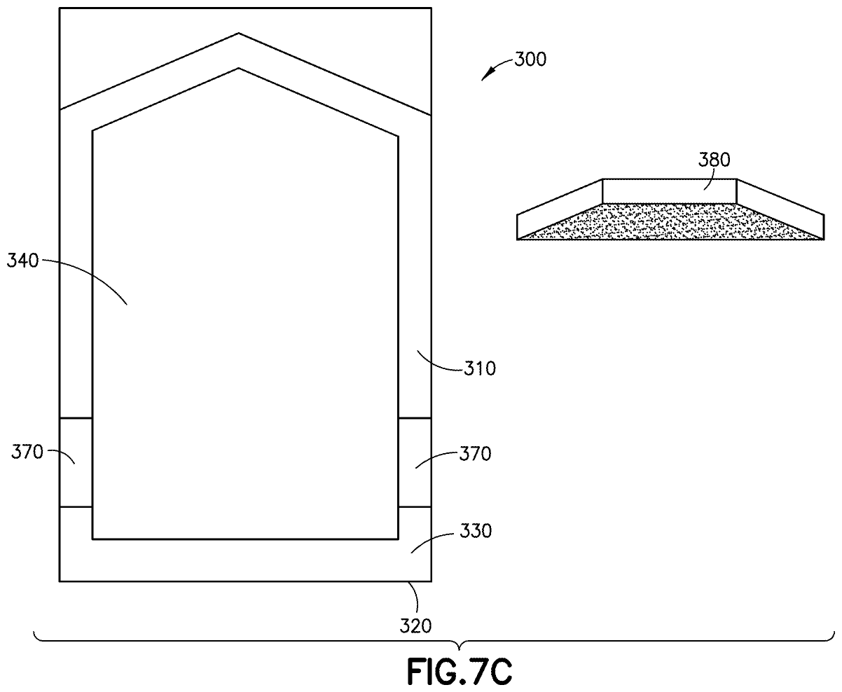

Referring to FIGS. 7A-7C, a terminally-sterilizable package 300 for a medical device according to non-limiting embodiments or aspects has a first web 310 in the form of a tray, optionally a tray formed of a rigid material, having a closed distal end, an open proximal end, and a sidewall 312 arranged therebetween defining a well 340 configured to receive the medical device. First web 310 can also include a flange 315 arranged about the perimeter of the open proximal end thereof. Second flexible web 320 can be arranged at the open proximal end of first flexible web 310, at least partially overlapping flange 315.

As shown in FIGS. 7A-7C, in non-limiting embodiments or aspects, sidewall 312 and/or flange 315 can include one or more cutouts 370. In non-limiting embodiments or aspects, package 300 includes one or more plugs 380 arranged within the one or more cutouts. One or more plugs 380 can be formed of a resilient and/or porous material, such as foam, and/or a sintered polyolefinic material. In non-limiting embodiments or aspects, one or more plugs 380 define a tortuous path fluidly connecting the well 340 with the exterior of the package 300. In non-limiting embodiments or aspects, one or more plugs 380 are larger than one or more cutouts 370, to provide a seal between one or more plugs 380 and sidewall 312 and/or flange 315. One or more cutouts 370 and plugs 380 can assume any useful configuration, but can, in non-limiting embodiments or aspects, have a circular cross-sectional shape or that of a quadrilateral, for example a rectangle, square, or trapezoid. Those of skill will appreciate that plugs, such as those described above, can be included in any other described embodiment, for example as a substitute for and/or addition to sealing regions (e.g., those defining tortuous paths as described herein).

In non-limiting embodiments or aspects, package 300 can include both cutouts and plugs 370, 380, as well as one or more tortuous paths (not shown) arranged between first web 310 and second flexible web 320 in one or more sealing regions 330, as described above with regard to FIGS. 1-6. Tortious paths useful in the illustrated non-limiting embodiment may have any characteristic of tortious paths 160, 260 described above.

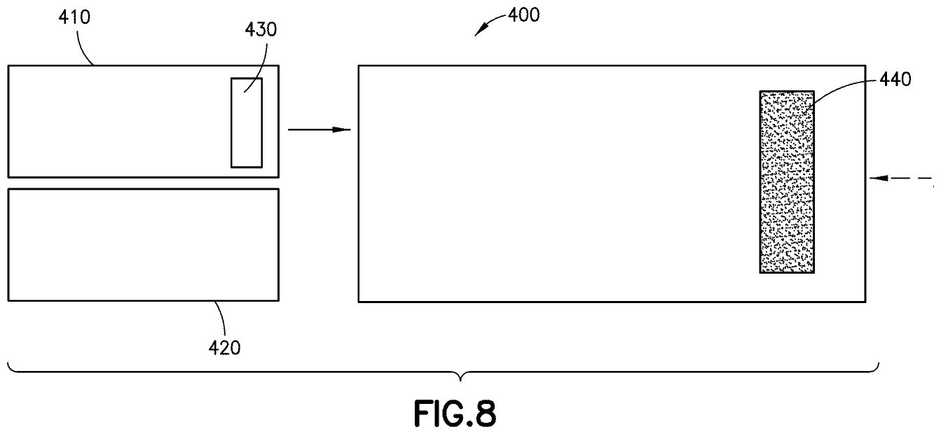

Turning to FIG. 8, shown is a non-limiting embodiment or aspect of package 400, including first and second flexible webs 410, 420 defining a compartment for holding, for example, a medical device. Package 400 may be terminally sterilized by a sterilizing gas and/or by irradiation. One or both of first and second flexible webs 410, 420 may include a cutout 430, in which a filter 440 may be placed. Edges of first and second flexible webs 410, 420 may be formed of one or more materials as described previously, and may be fused as described previously. In non-limiting embodiments or aspects, first and second flexible webs 410, 420, as well as filter 440, are formed of the same class of material, optionally the same material, enabling single-stream recycling of package 400. In non-limiting embodiments or aspects, first flexible web 410 and second flexible web 420 overlap at least partially at one or more regions along their respective perimeters, as described previously. Within these one or more regions of overlap are one or more sealing regions and/or plugs, as previously described. These one or more sealing regions may define one or more tortuous paths (for example, those shown in FIGS. 5A-5I) that fluidly connect the compartment and the exterior of the package 400. Tortious paths useful in the illustrated non-limiting embodiment may have any characteristic of tortious paths 160, 260 described above.

Filter 440 may serve a similar function as the tortuous paths and plugs described above, in that it may allow for passage of a sterilizing gas (e.g, hydrogen peroxide gas, EtO) to the compartment, sterilizing the interior thereof, including any medical devices placed therein. In addition, filter 440 may restrict the passage of microbes, thus maintaining sterility of the compartment, and any medical devices placed therein, following sterilization. Filter 440 may be formed of polypropylene, polyethylene, a sintered polyolefinic material, and/or like materials known to those of skill in the art as being useful for filtering air, and can have any useful characteristics, such a porosity, to maximize the ability for a sterilizing gas to pass therethrough while limiting ingress of microbes into the compartment. Use of such materials allows for the package 400 to be recycled in single-stream recycling. While window 430 and filter 440 are shown in FIG. 8 as being in the shape of a parallelogram, those of skill will appreciate that any suitable shape and size (including thickness) may be used. Filter 440 may be attached to first and/or second flexible webs 410, 420 through known techniques, such as, for example, heat staking, adhesives, ultrasonic welding, and/or laser welding.

Also provided herein is a method of making a terminally-sterilizable container as described herein. In non-limiting embodiments or aspects, filter 440 is attached to first and/or second flexible webs 410, 420, for example using one or more techniques described herein. Thereafter, first flexible web 410 and second flexible web 420 are attached to one another, for example using one or more techniques described herein, along one or more, but not all, of any areas of overlap. A medical device is then inserted into the compartment defined by the first and second flexible webs 410, 420, and the final area(s) of overlap are attached to one another, for example using one or more techniques described herein. Thereafter, package 400 is terminally sterilized as described herein.

Although the invention has been described in detail for the purpose of illustration based on what is currently considered to be the most practical and preferred embodiments, it is to be understood that such detail is solely for that purpose and that the invention is not limited to the disclosed embodiments, but on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A terminally-sterilizable package for a medical device, comprising:
a first flexible web having a proximal end, a distal end, and at least one side therebetween defining a first perimeter; and
a second flexible web having a proximal end, a distal end, and at least one side therebetween defining a second perimeter at least partially overlapping with the first perimeter at one or more sealing regions, the second flexible web removably attached to the first flexible web, the first flexible web and the second flexible web defining a compartment configured to hold the medical device,
wherein the one or more sealing regions define a tortuous path fluidly connecting the compartment with an exterior of the package.

2. The terminally-sterilizable package of claim 1, wherein the one or more sealing regions are arranged between the first flexible web and the second flexible web at the proximal ends thereof.

3. The terminally-sterilizable package of claim 1, wherein the one or more sealing regions are arranged between the first flexible web and the second flexible web at the distal ends thereof.

4. The terminally-sterilizable package of claim 1, wherein the one or more sealing regions are arranged between the first flexible web and the second flexible web at the one or more sides thereof.

5. The terminally-sterilizable package of claim 1, wherein the one or more sealing regions are arranged at more than one location of overlap between the first perimeter and the second perimeter.

6. The terminally-sterilizable package of claim 1, wherein the first flexible web and the second flexible web are formed of the same material.

7. The terminally-sterilizable package of claim 6, wherein the material comprises one or more flexible thermoformable plastics.

8. The terminally-sterilizable package of claim 7, wherein the flexible thermoformable plastics comprise one or more of nylon-based films, polyethylene, and/or ethyl vinyl acetate.

9. The terminally-sterilizable package of claim 1, wherein the tortuous path comprises one or more curves.

10. The terminally-sterilizable package of claim 1, wherein the tortuous path comprises one or more right angles.

11. The terminally-sterilizable package of claim 1, wherein the tortuous path comprises a honeycomb arrangement.

12. The terminally-sterilizable package of claim 1, wherein the tortuous path fluidly connects the compartment with the exterior of the package at a plurality of locations along the first perimeter and the second perimeter.

13. The terminally-sterilizable package of claim 1, wherein the tortuous path is arranged in substantially all of the overlap between the first perimeter and the second perimeter.

14. The terminally-sterilizable package of claim 1, wherein the first flexible web and the second flexible web comprise one or more overlapping regions extending radially inwards into the compartment.

15. The terminally-sterilizable package of claim 14, wherein the tortuous path is arranged at least in the one or more inwardly-extending regions.

16. A terminally-sterilizable package for a medical device, comprising:
a tray comprising:
a closed distal end;
an open proximal end;
a sidewall arranged between the closed distal end and the open proximal end, the closed distal end and sidewall defining a well configured to receive the medical device therein; and
a flange arranged about a perimeter of the open proximal end of the tray; and
a flexible web arranged at the open proximal end of the tray and at least partially overlapping the flange at one or more sealing regions,
wherein the one or more sealing regions define a tortuous path fluidly connecting the well with an exterior of the package.

17. The terminally-sterilizable package of claim 16, wherein the one or more sealing regions are arranged at more than one location of overlap between the flange and the flexible web.

18. The terminally-sterilizable package of claim 16, wherein the tray and/or the flexible web comprise one or more flexible thermoformable plastics.

19. The terminally-sterilizable package of claim 18, wherein the flexible thermoformable plastics comprise one or more of nylon-based films, polyethylene, and/or ethyl vinyl acetate.

20. The terminally-sterilizable package of claim 16, wherein the tortuous path comprises one or more curves.

21.-51. (canceled)
